(19) United States
(12) Patent Application Publication
(10) Pub. No.: US 2018/0338859 A1
(43) Pub. Date: Nov. 29, 2018

(54) OPHTHALMIC ENDOILLUMINATOR

(71) Applicant: Novartis AG, Basel (CH)

(72) Inventors: Alireza Mirsepassi, Irvine, CA (US); Michael J. Papac, North Tustin, CA (US)

(21) Appl. No.: 15/973,909

(22) Filed: May 8, 2018

Related U.S. Application Data
(60) Provisional application No. 62/510,329, filed on May 24, 2017.

(51) Int. Cl. A61F 9/007; F21V 8/00; G02B 23/24; A61B 1/07

(52) U.S. Cl. CPC A61F 9/007; G02B 6/0008; G02B 23/2469; A61B 2017/3454; A61B 2217/005; A61B 2217/007; A61B 1/07

(57) ABSTRACT

A method and system provide an endoilluminator including a hand piece, a needle coupled with the hand piece and an optical fiber coupled with the needle. The needle has a channel therethrough and an outside diameter. The channel has an inside diameter that is smaller than the outside diameter. At least a portion of the optical fiber coupled with the needle has a diameter of not more than one half the inside diameter.

This application claims the benefit of priority of U.S. Provisional Patent Application Ser. No. 62/510,329 titled “Ophthalmic Endoilluminator”, filed on May 24, 2017, whose inventors are Alireza Mirsepassi and Michael J. Papac, which is hereby incorporated by reference in its entirety as though fully and completely set forth herein.

### BACKGROUND

Ophthalmic surgery for the back of the eye (e.g. in the vitreous humor) frequently involves multiple ports, or apertures, into the eye through which various instruments are inserted. For example, three incisions through the sclera may be made. One port is used for the infusion cannula, through which a BSS® balanced salt solution is injected into the eye. This aids in maintaining the internal eye pressure during surgery. A needle for an endoilluminator is inserted through the second port. An endoilluminator typically includes a handpiece and a needle having a conventional optical fiber therein. The conventional optical fiber is typically formed of plastic and has a diameter on the order of 400-500 micrometers. This size is very close to the inside diameter of the needle. The conventional optical fiber provides illumination for the surgical field. The third port is used for a vitrectomy probe or other probe which may be used to remove unwanted material or otherwise perform procedures on the eye.

Although the ophthalmic surgery may be performed, there are possible negative outcomes that increase with the number of ports used. Having three ports requires the surgeon to make three incisions in the sclera and to monitor surgical instruments at three different locations. This makes the procedure more difficult for the surgeon. Three ports also carries a higher risk of complications and other negative outcomes for the patient than fewer ports. Additional ports might be used for various purposes. These additional ports may also make surgery more difficult for the physician and increase the risks for the patient.

Accordingly, what is needed is a mechanism for assisting a physician in ophthalmic surgery.

### BRIEF SUMMARY

A method and system provide an endoilluminator including a hand piece, a needle coupled with the hand piece and an optical fiber coupled with the needle. The needle has a channel therethrough and an outside diameter. The channel has an inside diameter that is smaller than the outside diameter. An optical fiber is coupled with the needle. A least a portion of the optical fiber coupled with the needle has a diameter of not more than one half the inside diameter. In some embodiments, this diameter may not be more than one hundred micrometers. Consequently, the endoilluminator may be used both for infusion or aspiration and as an endoilluminator. The endoilluminator may thus be an infusing or aspirating endoilluminator. In some embodiments, the optical fiber may be centrally mounted in the endoilluminator (e.g., extending close to the center of the endoilluminator). In some embodiments, the optical fiber may be mounted off-center or even along a side of the endoilluminator.

According to the method and system disclosed herein a single instrument may provide both illumination and infusion or aspiration, allowing for fewer ports and the possibility of improved patient outcomes.

### BRIEF DESCRIPTION OF SEVERAL VIEWS OF THE DRAWINGS

FIGS. 1A-D are diagrams depicting an exemplary embodiment of an endoilluminator and the endoilluminator used in conjunction with a console.

FIGS. 2A-B are diagrams depicting exemplary embodiments of an endoilluminator used in conjunction with a console.

FIGS. 3A and 3B are side and end views of another exemplary embodiment of an endoilluminator.

FIGS. 4A and 4B are side and end views of another exemplary embodiment of an endoilluminator.

FIGS. 5A and 5B are side and end views of another exemplary embodiment of an endoilluminator.

FIGS. 6A and 6B are side and end views of another exemplary embodiment of an endoilluminator.

FIGS. 7A and 7B are side and end views of another exemplary embodiment of an endoilluminator.

FIG. 8 depicts an exemplary embodiment of an optical fiber usable in an endoilluminator.

FIG. 9 depicts another exemplary embodiment of an optical fiber usable in an endoilluminator.

FIG. 10 depicts another exemplary embodiment of an optical fiber usable in an endoilluminator.

FIG. 11 is a flow chart depicting an exemplary embodiment of a method for providing an endoilluminator.

FIG. 12 is a flow chart depicting an exemplary embodiment of a method for assisting a physician using an endoilluminator.

### DETAILED DESCRIPTION

The exemplary embodiments relate to surgical instruments, such as those used in ophthalmic surgery. The following description is presented to enable one of ordinary skill in the art to make and use is the various embodiments that are provided in the context of a patent application and its requirements. Various modifications to the exemplary embodiments and the generic principles and features described herein will be readily apparent. The exemplary embodiments are mainly described in terms of particular methods and systems provided in particular implementations. However, the methods and systems will operate effectively in other implementations. Phrases such as “exemplary embodiment”, “one embodiment” and “another embodiment” may refer to the same or different embodiments as well as to multiple embodiments. The embodiments will be described with respect to systems and/or devices having certain components. However, the systems and/or devices may include more or less components than those shown, and variations in the arrangement and type of the components may be made without departing from the scope of the disclosure. The exemplary embodiments will also be described in the context of particular methods having certain elements. However, the method and system operate effectively for other methods having different and/or additional elements and elements in different orders that are not inconsistent with the exemplary embodiments. Thus, the present disclosure is not intended to be limited to the embodiments shown, but is to be accorded the widest scope consistent with the principles and features described herein.

The method and system are also described in terms of singular items rather than plural items. For example, an optical fiber is used and/or shown in some embodiments. One of ordinary skill in the art will recognize that these singular terms encompass plural. For example, multiple optical fibers might be used.

A method and system provide an endoilluminator including a hand piece, a needle coupled with the hand piece and an optical fiber coupled with the needle. The needle has a channel therethrough and an outside diameter. The channel has an inside diameter that is smaller than the outside diameter. The optical fiber is coupled with the needle. At least a portion of the optical fiber coupled with the needle has a diameter of not more than one half the inside diameter. In some embodiments, this diameter of the optical fiber may not be more than one hundred micrometers. Consequently, the endoilluminator may be used both for infusion or aspiration and as an endoilluminator. The endoilluminator may thus be an infusing or aspirating endoilluminator.

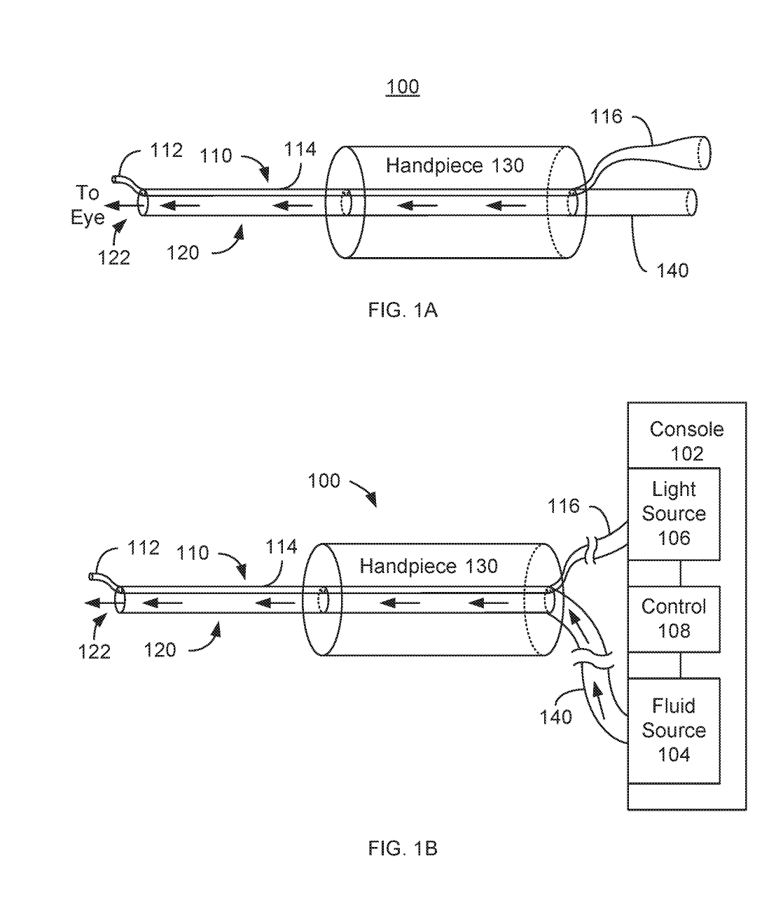

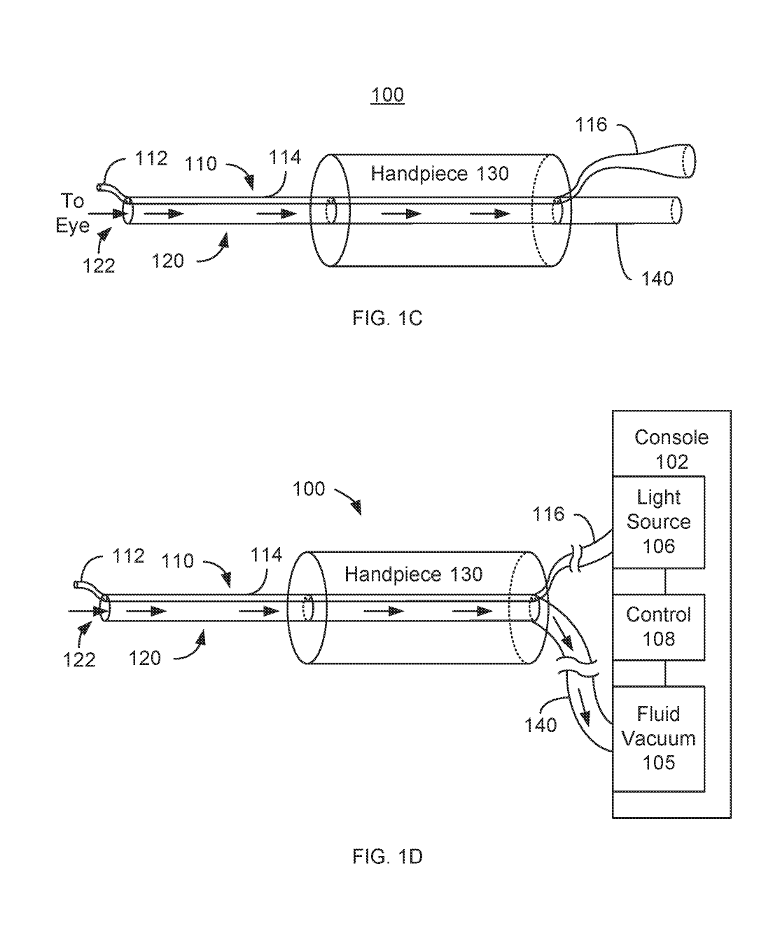

FIGS. 1A-D are diagrams depicting an exemplary embodiment of an infusing or aspirating endoilluminator 100 usable in ophthalmic surgery and the infusing or aspirating endoilluminator 100 when used in conjunction with a console 102. FIGS. 1A-D are not to scale and for explanatory purposes only. Thus, a particular infusing or aspirating endoilluminator is not intended to be shown.

The endoilluminator 100 includes an optical fiber 110, a needle 120 and a handpiece 130. Also shown is a fluid line 140 (for infusion or aspiration) that may be coupled with the needle 120. In some embodiments, the fluid line 140 may be integrated with the remainder of the hand piece 100. Although described as a fluid line 140, the fluid line 140 may also be known as an irrigation, infusion, or aspiration line.

The handpiece 130 is coupled with the needle 120. For simplicity, the handpiece 130 is shown as cylindrical. However, the handpiece 130 may have another, typically more complex, shape. In addition, the handpiece 130 may also have other components with other functions that are not shown for simplicity. The handpiece 130 may be used to manipulate the needle 120 and thus the optical fiber 110.

In addition to being coupled with the handpiece 130, the needle 120 may be connected with the fluid line 140. The needle 120 is hollow, having a channel 122 therein. In the embodiment shown, the channel 122 is along the axis of the needle 120. Therefore, although not explicitly indicated in FIGS. 1A-D, the needle has an inside diameter that is the diameter of the channel and an outside diameter. The inside diameter is less than the outside diameter by at least the thickness of the walls of the needle 120. A fluid such as a BSS® may flow from the fluid line 140, through the channel 122 and into the eye. This flow of fluid is indicated in FIGS. 1A-B by arrows passing through the channel 122.

Consequently, the fluid flow through the needle 120 may be used to maintain the eye pressure during a procedure. In some embodiments, fluid may be aspirated through the channel 122 and into fluid line 140 (e.g., during membrane peeling or dissection). This flow of fluid is indicated in FIGS. 1C-D by arrows passing through the channel 122 to the fluid vacuum 105.

The optical fiber 110 is coupled with the needle 120. In the embodiment shown, at least part of the optical fiber is within the channel 122. Also in the embodiment shown, the optical fiber 110 lies along the wall of channel 122. In other embodiments, the optical fiber 110 may be at another location. For example, the optical fiber may be along the axis of the needle 110 or in another position in the channel 122. Alternatively, the optical fiber 122 may be along the outside of the needle 120. The optical fiber 110 is also shown as being parallel to the axis of the channel 122. In other embodiments, the optical fiber 110 may be curved or retained within or around the needle 120 in another manner.

The diameter of at least a portion of the optical fiber 110 is small in comparison to the diameter of the channel 122. In some embodiments, the portion of the optical fiber 110 within the channel 122 has a diameter that may not be more than one-half the inside diameter of the channel 122. In some embodiments, the diameter of the optical fiber 110 may not be more than one hundred micrometers. In some such embodiments, at least the portion of the optical fiber 110 within the channel 122 has a diameter of not more than sixty micrometers. This diameter of the optical fiber 110 may be not more than fifty micrometers in some cases. For example, at least the portion of the optical fiber 110 within the channel may have a diameter of less than fifty micrometers and at least thirty micrometers. Some or all of the optical fiber 110 may have such a small diameter even if the optical fiber 110 lies along the needle 110, outside of the channel 122.

In some embodiments, the optical fiber 110 is a fused silica (e.g. glass) fiber or a borosilicate fiber. In other embodiments, only the portion of the optical fiber 110 having the reduced diameter discussed above is formed of fused silica and/or borosilicate. Plastic or other materials that cannot withstand the heat load might not be used for the reduced diameter portion of the optical fiber 110, such as portions 112 and 114. This allows the optical fiber 110 to withstand the heat generated by light transmitted through the optical fiber 110. However, in other embodiments, additional or different material(s) may be used.

The optical fiber 110 may be formed by heating and drawing a fiber that initially has a larger diameter. Such a fiber may also be tapered. One such embodiment is shown in FIGS. 1A-D. The optical fiber 110 has portions 112 and 114 having the diameter described above. Another portion 116 may have a larger diameter. Thus, portions 112 and 114 may be formed by drawing an optical fiber having a diameter at least as large as that of the portion 116. Such a fiber may be heated using a laser and pulled to provide an optical fiber at least part of which has the smaller diameter described above. In other embodiments, the optical fiber consists of small diameter portions 112 and 114, but is coupled with another larger-diameter fiber 116 that may be tapered. Portion 112 of the optical fiber 110 extends out of the channel 122 of the needle 120. Thus, the tip of the optical fiber 110 is outside of the channel 122. However, in other embodiments, the optical fiber 110 terminates at the tip/end of or within the channel 122 of the needle 120.

In the embodiment shown in FIGS. 1A-D, the tip of the optical fiber 110 is flat cleaved. In other embodiments, the tip of the optical fiber 110 may be configured in another manner. For example, the tip of the optical fiber 110 may be tapered, have a scattering tip or otherwise shaped to provide the desired illumination. Because it has a small diameter and may not be unduly lossy, the optical fiber 110 may concentrate the light input to the end 116. This may not only increase the temperature of the optical fiber 110, but also the intensity of the light output by the optical fiber 110. However, the more intense light illuminates a smaller area. Consequently, a tapered or scattering tip which directs light in multiple directions may be desired to increase the field that is illuminated. If the flat cleaved tip shown is used, then the launch half-angle of the beam into the optical fiber 110 may be increased in order to augment scattering. For example, the launch half-angle may be at least thirty degrees from the axis of the fiber 116. Thus, a high numerical aperture may also be desired for the optical fiber 110. Optical fiber 110 can also have a tapered section in the middle with tip being flat cleaved. The taper increases the angular content of light before it reaches out to flat cleaved tip. Alternatively, the tip of the optical fiber 110 may be shaped or have another component used to increase the size of the area illuminated.

The endoilluminator 100 (for infusion) is shown in use in FIGS. 1A-B. The endoilluminator 100 (for aspirating) is shown in use in FIGS. 1C-D. The endoilluminator 100 is coupled with the console 102. The console 102 includes a fluid source 104 and/or fluid vacuum 105, control block 108 and light source 106. In other embodiments, the fluid source 104 (and/or fluid vacuum 105) and/or light source 106 may be physically separated from the console. The control block 108 may include a processor executing instructions stored in a memory and capable of communicating an input/output device such as a graphical user interface (GUI) or other mechanism that the user employs to control the fluid flow and light. The fluid source 104 and/or fluid vacuum 105 is coupled with the needle 120 via the fluid line 140, which may be formed of tubing. The fluid source 104 may be a source of BSS®. The BSS® in the fluid source 104 can be placed under a positive pressure and driven through the fluid line 140. In some embodiments, the fluid vacuum 105 may pull a vacuum through the fluid line 140 to aspirate fluid through the endoilluminator 100. For example, the fluid vacuum 105 may be, a venturi pump, a peristalitic pump, etc. The light source 106 is coupled with the thin portions 112 and 114 of the optical fiber via portion 116. As discussed above, some or all of the portion 116 may be formed of a different optical fiber coupled to the optical fiber 110. A user, such as a surgeon, may control the illumination, fluid, and/or other electronics for the endoilluminator 100 using the console 102.

In operation, the needle 120 may be inserted into an incision in the eye to perform ophthalmic surgery. The surgeon may make additional incision(s) for other purposes. Fluid may flow from the fluid source 104 through fluid line 140 and through the channel 122 of the needle 120. The arrows in FIGS. 1A-D within the fluid line 140 and needle 120 depict the direction of fluid flow. Because the diameter of the optical fiber 110 is significantly smaller than the diameter of the channel 122, the optical fiber occupies such a small portion of the cross section of the channel 122. This allows the fluid to flow relatively freely through the needle 120. Consequently, eye pressure may be maintained.

In addition, light from the light source 106 is coupled into the optical fiber 110. As discussed above, the light may be scattered from the tip of the optical fiber 110 in order to illuminate a larger region. As a result, the endoilluminator may not only illuminate the operating field, but also provide fluid (or aspirate fluid) to/from the eye.

The infusing or aspirating endoilluminator 100 may result in improved outcomes. Because both illumination and fluid are provided (or fluid may be aspirated while illumination is provided) via a single device 100, only one incision may be made in the eye to provide light and maintain eye pressure or aspirate fluid from the eye. For example, a surgery previously requiring three ports may use not more than two ports. Similarly, a surgery previously utilizing four ports may need at most three ports. Fewer incisions carry reduced risk of complications to the patient. Furthermore, the surgeon must monitor fewer incisions. Consequently, surgery may be made simpler, faster and less error prone. Because the optical fiber 110 is sufficiently thin for fluid to flow more freely in the channel 122, a high pressure for the fluid line may not be necessary. The optical fiber 110 is sufficiently robust to preclude or reduce damage from heat generated by the light carried in the optical fiber 110. Thus, the risk to the patient and ease of surgery are further improved.

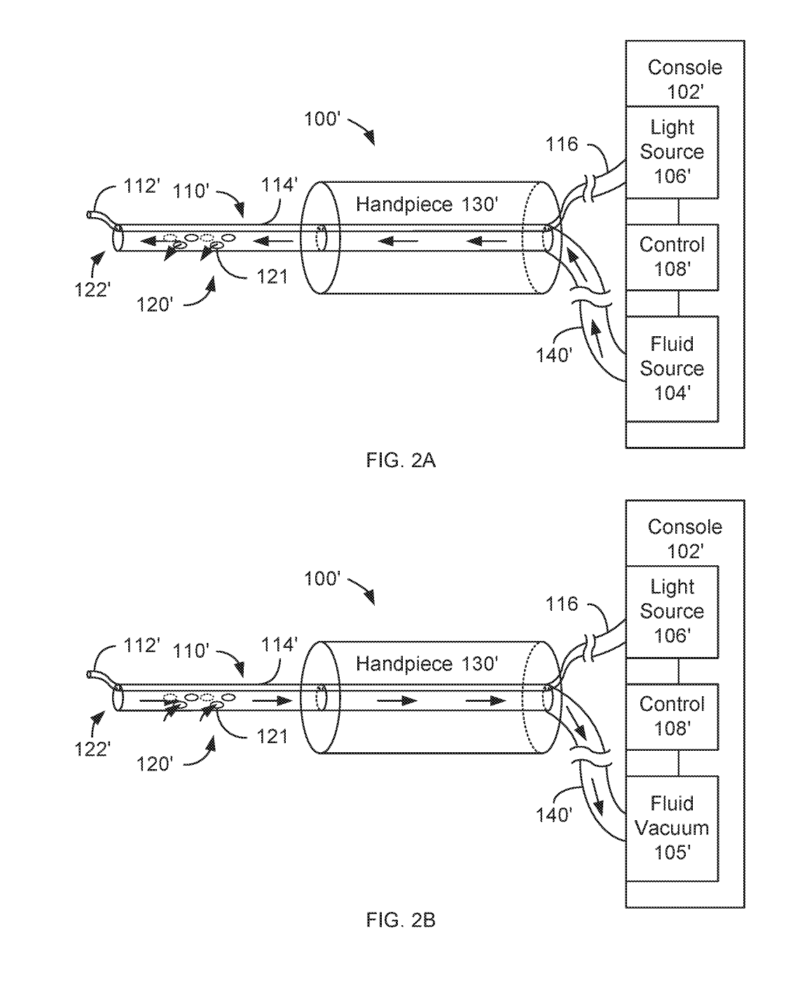

FIGS. 2A-B depict additional exemplary embodiments of an endoilluminator 100′ as used with console 102′. FIGS. 2A-B are not to scale and for explanatory purposes only. Thus, a particular endoilluminator is not intended to be shown. In addition, only some portions of the endoilluminator 100′ are shown. The endoilluminator 100′ and console 102′ are analogous to the endoilluminator 100 and console 102, respectively, depicted in FIGS. 1A-D. The optical fiber 110′, needle 120′ and handpiece 130′ depicted in FIGS. 2A-B are analogous to the optical fiber 110, needle 120 and handpiece 130 of FIGS. 1A-D. Similarly, the fluid line 140′, fluid source 104′, fluid vacuum 105′, light source 106′ and control block 108′ are analogous to the fluid line 140, fluid source 104, fluid vacuum 105, light source 106 and control block 108 depicted in FIGS. 1A-D. The portions 112′, 114′ and 116′ of the optical fiber 110′ and the channel 122′ of the needle 120′ are analogous to the portions 112, 114 and 116 of the optical fiber 110 and the channel 122 of the needle 120, respectively.

In addition, the needle 120′ also includes apertures 121. For clarity, only one aperture 121 is labeled. The apertures 121 are shown in a particular configuration. However, other configurations might be used. The apertures 121 may reside along only a portion of the needle 120′ as shown or may reside along the entire channel 122′ that is outside of the handpiece 130′. The apertures 121 may reside around the outside surface of the needle 120′ or only along one side of the needle 120′. Although shown as distributed, the apertures 121 may be aligned along a particular line. Although multiple apertures 121 are shown, the needle 120′ may include another number including a single aperture.

The apertures 121 allow fluid from the fluid source 104′ to exit the channel 122′ at locations other than the distal end (at which the fiber 110′ exits the needle 120′). In some embodiments, the distal end of the channel 122′ may be closed to fluid. In such embodiments, fluid may be provided only through the apertures 121. Only illumination from the optical fiber 110′ and/or the optical fiber 110′ itself to exit the end of the needle 120′. In some embodiments, fluid may flow from both the distal end of the needle 120′ and the apertures 121. In some embodiments, fluid may be aspirated from the distal end of the needle 120′ or from both the distal end of the needle 120′ and the apertures 121.

The endoilluminator 100′ may share the benefits of the endoilluminator 100. The presence of the apertures 1221 may provide additional benefit(s). Apertures 121 allow fluid to be provided or aspirated to/from the eye with a lower (or zero) pressure near the end of the needle 120′. Such a configuration may be useful in situations in which a fragile structure, such as the retina, is desired to be illuminated. Light from the optical fiber 110′ may be provided to the structure without the risk of the structure being damaged by fluid flowing or being aspirated to/from the end of the needle 120′. Thus, use of the endoilluminator may be extended to procedures in which fragile structures are to be illuminated.

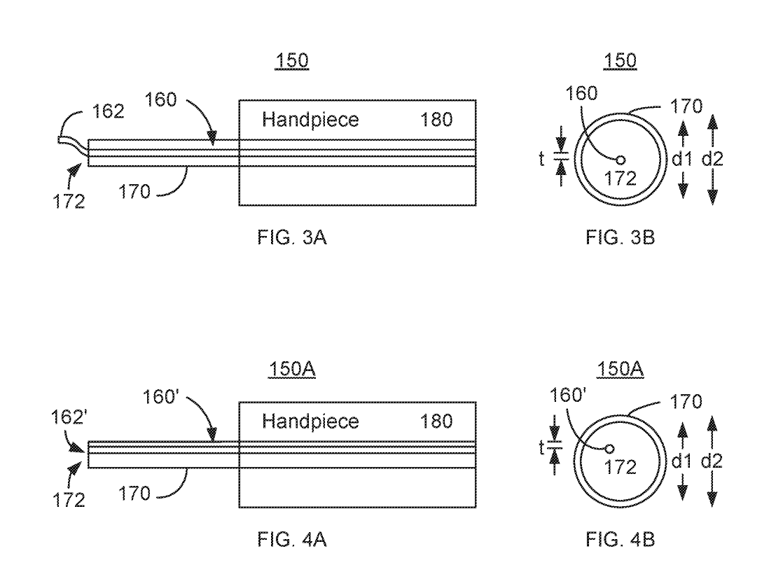

FIG. 3A depicts a side view of another exemplary embodiment of an endoilluminator 150 usable in ophthalmic surgery. FIG. 3B is a cross-sectional view of a portion of the endoilluminator. FIGS. 3A-B are not to scale and for explanatory purposes only. Thus, a particular endoilluminator is not intended to be shown. In addition, only some portions of the endoilluminator 150 are shown.

The endoilluminator 150 includes an optical fiber 160, a needle 170 and a handpiece 180 that are analogous to the optical fiber 110, needle 120 and handpiece 130, respectively. The needle 170 is hollow and has a channel 172 that is parallel to the axis and analogous to the channel 122. The inside diameter of the needle 170, d1, is the diameter of the channel 172. The outside diameter of the needle 170 is d2. In some embodiments, the inside diameter d1 is on the order of three hundred through five hundred micrometers. However, other sizes are possible.

The optical fiber 160 is analogous to the optical fiber 110 and may have similar diameters. Thus, the diameter, t, of the optical fiber 160 may not be more than one half of the diameter of the channel 172. In some embodiments, t may not be more than one third of d1. In some such embodiments, the optical fiber diameter may not be more than one hundred micrometers. In other embodiments, the diameter of the optical fiber 160 may not be more than sixty micrometers. The diameter of the optical fiber 160, t, may be not more than fifty micrometers. In some cases, t may be at least thirty micrometers. The diameter of the optical fiber 160 is such that the optical fiber 160 can transmit the desired amount of light without heat induced damage while being sufficiently small to allow fluid to flow through the channel 172. As can be seen in FIGS. 3A-B, the optical fiber 160 is along the axis (center) of the channel 172. However, other locations may be possible. In some embodiments, the optical fiber 160 may be secured to a central portion at the proximal end of the needle 170 (the end closest to the handle). For example, the optical fiber 160 may emerge from a sleeve (e.g., a sleeve secured relative to the needle 170) in the central portion of the needle 170. The optical fiber 160 may be rigid such that the optical fiber 160 tends to stay toward the center of the needle 170 even as the optical fiber 160 extends toward the distal end of the needle 170. Further, the optical fiber may extend down the needle off of an interior channel wall such that fluid flowing through the needle 170 (either being infused or aspirated) circumscribes the optical fiber 160. In some embodiments, fluid flow around the optical fiber 160 may further reinforce the optical fiber's position in the center (i.e., fluid flow may bias the optical fiber 160 toward the center of the needle 170 as the fluid flows through the needle 170 on all sides of the optical fiber 160). A portion 162 of the optical fiber 160 protrudes from the needle 150. In other embodiments, the optical fiber 160 may terminate at the tip of or within the channel 172. Although a flat cleaved tip is shown for the optical fiber 160, other tips may be used.

The endoilluminator 150 shares the benefits of the endoilluminator 100. Both illumination and fluid are provided (or fluid may be aspirated while illumination is provided) via a single device 150. Thus, light may be provided and eye pressure maintained using only a single incision in the eye. Fewer incisions carry reduced risk of complications to the patient. Furthermore, surgery may be made simpler, faster and less error prone. Because the optical fiber 160 is sufficiently thin, the optical fiber 160 occupies a small fraction of the cross-sectional area of the channel 172. Thus, fluid may flow more freely in the channel 172 and a high pressure for the fluid line may not be necessary. The optical fiber 160 is sufficiently robust to preclude or reduce damage from heat generated by the light carried in the optical fiber 160. Thus, the risk to the patient and ease of surgery are further improved.

FIG. 4A depicts a side view of another exemplary embodiment of an endoilluminator 150A usable in ophthalmic surgery. FIG. 4B is a cross-sectional view of a portion of the endoilluminator 150A. FIGS. 4A-B are not to scale and for explanatory purposes only. Thus, a particular endoilluminator is not intended to be shown. In addition, only some portions of the endoilluminator 150A are shown.

The endoilluminator 150A includes an optical fiber 160′, a needle 170 and a handpiece 180 that are analogous to the optical fiber 160, needle 170 and handpiece 180, respectively. The needle 170 is hollow and has a channel 172. The inside diameter of the needle 170, d1, is the diameter of the channel 172. The outside diameter of the needle 170 is d2.

The optical fiber 160′ is analogous to the optical fiber 160 and may have similar diameters. Thus, t, d1 and d2 may be analogous to those described above. However, the optical fiber 160′ terminates at the end of the channel 172. In addition, the optical fiber 160′ is shown as residing off of the axis for the channel 172. Thus, there is no requirement that an optical fiber lie along the axis, against a wall of the channel 172 or at a particular location. In some embodiments, the optical fiber 160 may be secured to an off-axis portion at the proximal end of the needle 170 (the end closest to the handle). For example, the optical fiber 160 may emerge from a sleeve (e.g., a sleeve secured relative to the needle 170) in an off-axis portion of the needle 170. The optical fiber 160 may be rigid such that the optical fiber 160 tends to stay in the off-axis position of the needle 170 even as the optical fiber 160 extends toward the distal end of the needle 170. Further, the optical fiber may extend down the needle off of an interior channel wall such that fluid flowing through the needle 170 circumscribes the optical fiber 160. Although a flat cleaved tip is shown for the optical fiber 160′, other tips may be used.

The endoilluminator 150A shares the benefits of the endoilluminator 150. Both illumination and fluid are provided (or fluid may be aspirated while illumination is provided) via a single device 150A. Thus, only one incision may be made in the eye to provide light and maintain eye pressure or aspirate fluid from the eye. Fewer incisions carry reduced risk of complications to the patient and may make surgery simpler, faster and less error prone. Because the optical fiber 160 is sufficiently thin for fluid to flow more freely in the channel 172, a high pressure for the fluid line may not be necessary. The optical fiber 160′ is sufficiently robust to preclude or reduce damage from heat generated by the light carried in the optical fiber 160′. Thus, the risk to the patient and ease of surgery are further improved.

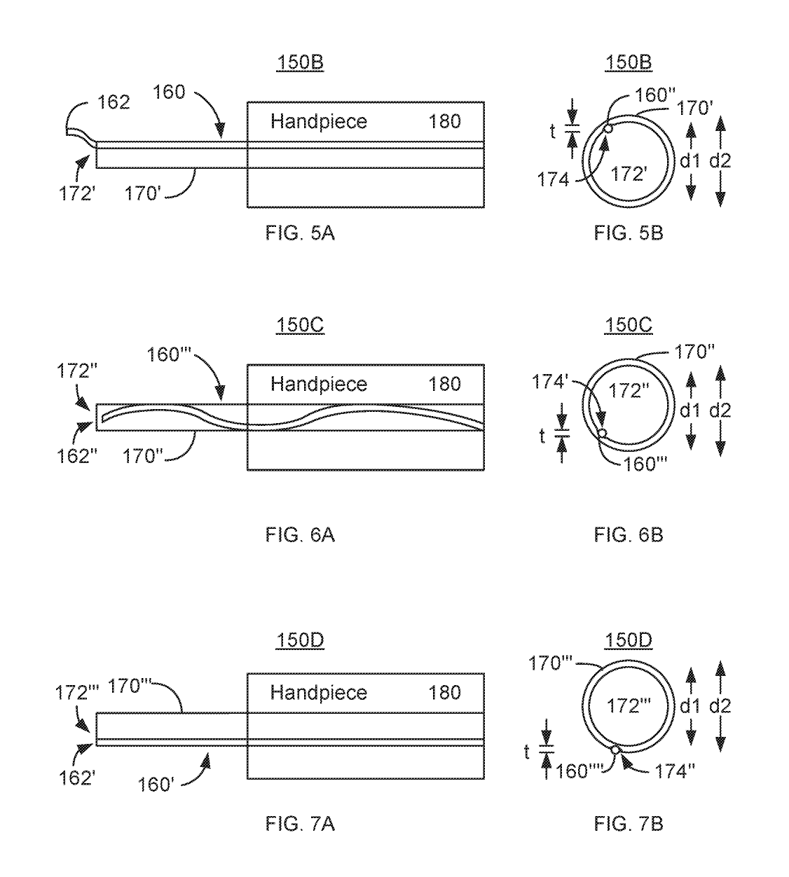

FIG. 5A depicts a side view of another exemplary embodiment of an endoilluminator 150B usable in ophthalmic surgery. FIG. 5B is a cross-sectional view of a portion of the endoilluminator 150B. FIGS. 5A-B are not to scale and for explanatory purposes only. Thus, a particular endoilluminator is not intended to be shown. In addition, only some portions of the endoilluminator 150B are shown.

The endoilluminator 150B includes an optical fiber 160″, a needle 170′ and a handpiece 180 that are analogous to the optical fiber 160 and/or 160′, needle 170 and handpiece 180, respectively. The needle 170′ is hollow and has a channel 172′. The inside diameter of the needle 170′, d1, is the diameter of the channel 172′. The outside diameter of the needle 170′ is d2. In addition, the channel 172′ includes a depression 174, or groove, in which the optical fiber 160″ resides. The groove 174 may be used to guide and retain the optical fiber 160″ in the channel 172′. In some embodiments, epoxy, resin or another substance may be used to hold the optical fiber 160″ in the groove 174. The epoxy may also serve to provide mechanical protection, optical isolation, heat sinking, or serve another purpose for the optical fiber 160″.

The optical fiber 160″ is analogous to the optical fibers 160 and/or 160′ and may have similar diameters. Thus, t, d1 and d2 may be analogous to those described above. The optical fiber 160 may terminate outside of the channel 172′ as shown or at another location. In addition, the optical fiber 160 is shown as residing at a particular location along the wall of the channel 172′, instead of being near the interior of the channel 172′. Other locations are possible. Although a flat cleaved tip is shown for the optical fiber 160″, other tips may be used.

The endoilluminator 150B shares the benefits of the endoilluminator 150 and/or 150A. Both illumination and fluid are provided (or fluid may be aspirated while illumination is provided) via a single device 150B. Consequently, a single incision in the eye may be used both to provide light and maintain eye pressure or aspirate fluid from the eye. Fewer incisions carry reduced risk of complications to the patient. Surgery may also be simpler and less error prone. Because the optical fiber 160″ occupies a small area of the cross section of the channel 172′, fluid may flow more freely in the channel 172′. A high pressure for the fluid line may not be necessary. The optical fiber 160 is sufficiently robust to preclude or reduce damage from heat generated by the light carried in the optical fiber 160″. Use of epoxy or an analogous substance may also improve the robustness of the optical fiber 160″. Thus, the risk to the patient, time taken for the surgery and ease of surgery are further improved.

FIG. 6A depicts a side view of another exemplary embodiment of an endoilluminator 150C usable in ophthalmic surgery. FIG. 6B is a cross-sectional view of a portion of the endoilluminator 150C. FIGS. 6A-B are not to scale and for explanatory purposes only. Thus, a particular endoilluminator is not intended to be shown. In addition, only some portions of the endoilluminator 150C are shown.

The endoilluminator 150C includes an optical fiber 160′″, a needle 170″ and a handpiece 180 that are analogous to the optical fiber 160, 160′ and/or 160″, needle 170 and/or 170′ and handpiece 180, respectively. The needle 170″ is hollow and has a channel 172″. The inside diameter of the needle 170″, d1, is the diameter of the channel 172″. The outside diameter of the needle 170″ is d2. In addition, the channel 172″ includes a depression 174′, or groove, in which the optical fiber 160″ resides. In this case, the groove 174 may not be parallel to the axis of the channel 172″. In some embodiments, epoxy, resin or another substance may be used to hold the optical fiber 160′″ in the groove 174′. The epoxy may also serve to provide mechanical protection, optical isolation, heat sinking or serve another purpose for the optical fiber 160′″.

The optical fiber 160′″ is analogous to the optical fibers 160, 160′, and/or 160″ and may have similar diameters. Thus, t, d1 and d2 may be analogous to those described above. The optical fiber 160′″ may terminate inside of the channel 172″ as shown or at another location. In addition, the optical fiber 160′″ is shown as residing at a particular location along the wall of the channel 172″. The optical fiber 160′″ also curves in its path along the channel 172″. However, other locations and paths are possible. Although a flat cleaved tip is shown for the optical fiber 160′″, other tips may be used

The endoilluminator 150C shares the benefits of the endoilluminator 150, 150A and/or 150B. Because both illumination and fluid are provided (or fluid may be aspirated while illumination is provided) via a single device 150C, only one incision may be made in the eye to provide light and maintain eye pressure or aspirate fluid from the eye. Fewer incisions carry reduced risk of complications to the patient and may make surgery simpler and less error prone. Because the optical fiber 160′″ is sufficiently thin for fluid to flow more freely in the channel 172″, a high pressure for the fluid line may not be necessary. The optical fiber 160′″ is sufficiently robust to preclude or reduce damage from heat generated by the light carried in the optical fiber 160′″. Use of epoxy or an analogous substance may also improve the robustness of the optical fiber 160′″. Thus, the risk to the patient, time taken for the surgery and ease of surgery are further improved.

FIG. 7A depicts a side view of another exemplary embodiment of an endoilluminator 150D usable in ophthalmic surgery. FIG. 7B is a cross-sectional view of a portion of the endoilluminator 150D. FIGS. 7A-7B are not to scale and for explanatory purposes only. Thus, a particular endoilluminator is not intended to be shown. In addition, only some portions of the endoilluminator 150D are shown.

The endoilluminator 150D includes an optical fiber 160″″, a needle 170′″ and a handpiece 180 that are analogous to the optical fiber 160, 160′, 160″ and/or 160′″, needle 170, 170′ and/or 170″ and handpiece 180, respectively. The needle 170′″ is hollow and has a channel 172′″. The inside diameter of the needle 170′″, d1, is the diameter of the channel 172′″. The outside diameter of the needle 170′″ is d2. In addition, the outside of the needle 170′″ includes a depression 174″, or groove, in which the optical fiber 160″ resides. In this case, the groove 174″ is parallel to the axis of the channel 172″. However, another configuration is possible. In some embodiments, epoxy, resin or another substance may be used to hold the optical fiber 160 in the groove 174″. The epoxy may also serve to provide mechanical protection, optical isolation, heat sinking or serve another purpose for the optical fiber 160″″. Because the optical fiber 160″″ is outside of the channel 172′″, more space in the channel is available for fluid flow.

The optical fiber 160″″ is analogous to the optical fibers 160, 160′, 160″ and/or 160′″ and may have similar diameters. Thus, t, d1 and d2 may be analogous to those described above. The optical fiber 160′″ may terminate at the end of the channel 172′″ as shown or at another location. In addition, the optical fiber 160′ is shown as residing at a particular location along the wall of the channel 172 and having a path parallel to the axis of the channel 172. Other locations and/or other paths are possible. Although a flat cleaved tip is shown for the optical fiber 160″″ other tips may be used

The endoilluminator 150D shares the benefits of the endoilluminator 150, 150A, 150B and/or 150C. Both illumination and fluid are provided (or fluid may be aspirated while illumination is provided) via a single device 150D. Thus, only one incision may be made in the eye to provide light and maintain eye pressure or aspirate fluid from the eye. Fewer incisions carry reduced risk of complications to the patient, may make surgery simpler and may allow surgery to be less error prone. Because the optical fiber 160″″ is outside of the channel 172′″ fluid may flow more freely in the channel 172″. Thus, a high pressure for the fluid line may not be necessary. The optical fiber 160′ is sufficiently robust to preclude or reduce damage from heat generated by the light carried in the optical fiber 160″″. Use of epoxy or an analogous substance may also improve the robustness of the optical fiber 160″″. Thus, the risk to the patient, time taken for the surgery and ease of surgery are further improved.

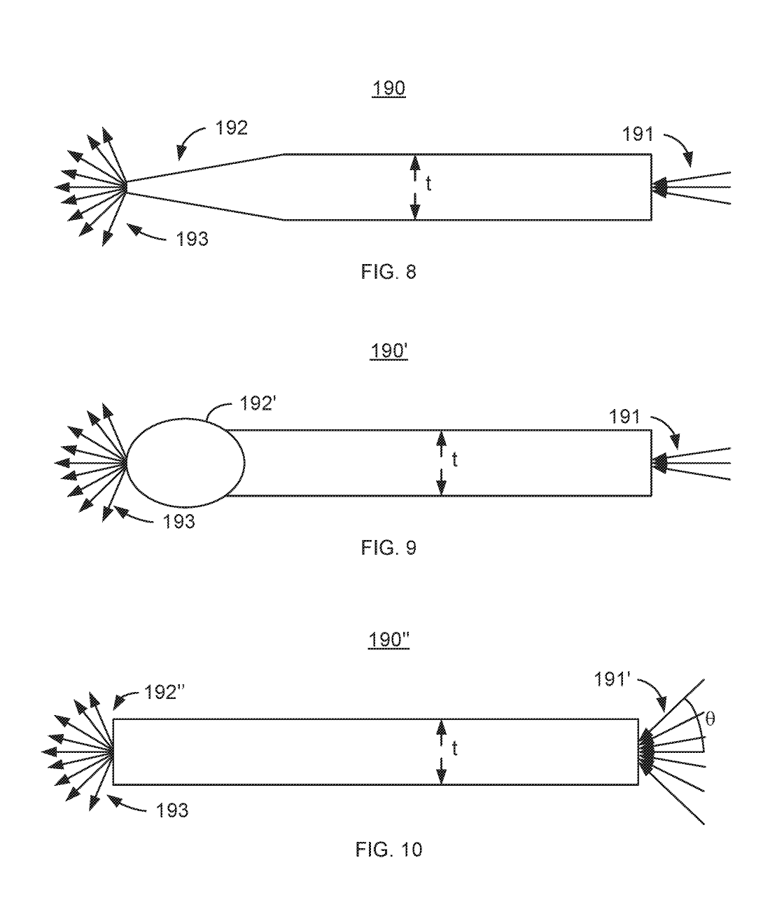

FIG. 8 depicts a side view of another exemplary embodiment of an optical fiber 190 that may be used in an endoilluminator usable in ophthalmic surgery. FIG. 8 is not to scale and for explanatory purposes only. Thus, a particular optical fiber is not intended to be shown. In addition, only some portions of the optical fiber 190 are shown. The optical fiber 190 may be used in an endoilluminator 100, 150, 150A, 150B, 150C, 150D and/or another endoilluminator. Also shown are input beam 191 and output light 193.

The optical fiber 190 is analogous to the optical fibers 110, 160, 160′, 160″, 160′″ and/or 160″″ and may have similar diameters. The diameter of the optical fiber 190 is t. Thus, t may be analogous to that described above. In addition, the optical fiber 190 has a tip 192. The tip 192 is tapered. As a result, the output light 193 may be more efficiently spread. Thus, light may illuminate a larger region.

The optical fiber 190 shares the benefits of the optical fiber 110, 160, 160′, 160″, 160′″ and/or 160″″ when used in an endoilluminator. Both illumination and fluid may be provided (or fluid may be aspirated while illumination is provided) via a single device. Fewer incisions may be made and surgery may be simpler. The optical fiber 190 may also be formed of silica or like materials to ensure they remain robust despite rises in temperature due to light carried in the fiber 190. The optical fiber 190 is sufficiently robust to preclude or reduce damage from heat generated by the light carried in the optical fiber 190. Thus, the risk to the patient and ease of surgery are further improved.

FIG. 9 depicts a side view of another exemplary embodiment of an optical fiber 190′ that may be used in an endoilluminator usable in ophthalmic surgery. FIG. 9 is not to scale and for explanatory purposes only. Thus, a particular optical fiber is not intended to be shown. In addition, only some portions of the optical fiber 190′ are shown. The optical fiber 190′ may be used in an endoilluminator 100, 150, 150A, 150B, 150C, 150D and/or another endoilluminator. Also shown are input beam 191 and output light 193.

The optical fiber 190′ is analogous to the optical fibers 110, 160, 160′, 160″, 160′″, 160″″ and/or 190 and may have similar diameters. The diameter of the optical fiber 190′ is d. Thus, t may be analogous to that described above. In addition, the optical fiber 190 has a tip 192′. The tip 192′ may more efficiently scatter light. Although an ellipsoid is shown for the tip 192′, the scattering tip 192′ may have another shape that scatters light. Thus, the output light 193 may illuminate a larger region.

The optical fiber 190′ shares the benefits of the optical fiber 110, 160, 160′, 160″, 160′″, 160″″ and/or 190 when used in an endoilluminator. Both illumination and fluid may be provided (or fluid may be aspirated while illumination is provided) via a single device. Fewer incisions may be made and surgery may be simpler. The optical fiber 190′ may also be formed of silica or like materials to ensure they remain robust despite rises in temperature due to light carried in the fiber 190′. The optical fiber 190′ is sufficiently robust to preclude or reduce damage from heat generated by the light carried in the optical fiber 190′. Thus, the risk to the patient and ease of surgery are further improved.

FIG. 10 depicts a side view of another exemplary embodiment of an optical fiber 190′ that may be used in an endoilluminator usable in ophthalmic surgery. FIG. 10 is not to scale and for explanatory purposes only. Thus, a particular optical fiber is not intended to be shown. In addition, only some portions of the optical fiber 190″ are shown. The optical fiber 190″ may be used in an endoilluminator 100, 150, 150A, 150B, 150C, 150D and/or another endoilluminator. Also shown are input beam 191′ and output light 193.

The optical fiber 190″ is analogous to the optical fibers 110, 160, 160′, 160″, 190 and/or 190′ and may have similar diameters. The diameter of the optical fiber 190″ is d. Thus, t may be analogous to that described above. In addition, the optical fiber 190 has a flat cleaved tip 192″. In order for the tip 192″ to more efficiently scatter light, the input light 191′ may be at a higher launch half-angle, θ. For example, θ may be at least thirty degrees and less than ninety degrees. The numerical aperture for the fiber 190″ may also be large. Thus, the output light 193 may illuminate a larger region. In an alternate embodiment, the optical fiber 190″ might be tapered in the middle to increase the angular content of light before reaching to the flat cleaved tip 192″. In such an embodiment, the input angle of the light may be less than thirty degrees.

The optical fiber 190″ shares the benefits of the optical fiber 110, 160, 160′, 160″, 160′″, 160″″, 190 and/or 190′ when used in an endoilluminator. Both illumination and fluid may be provided (or fluid may be aspirated while illumination is provided) via a single device. Fewer incisions may be made and surgery may be simpler. The optical fiber 190″ may also be formed of silica or like materials to ensure they remain robust despite rises in temperature due to light carried in the fiber 190″. The optical fiber 190″ is sufficiently robust to preclude or reduce damage from heat generated by the light carried in the optical fiber 190″. Thus, the risk to the patient and ease of surgery are further improved.

Various characteristics of endoilluminators and optical fibers have been shown. One of ordinary skill in the art will recognize that one or more of these features may be combined in manners not explicitly shown herein.

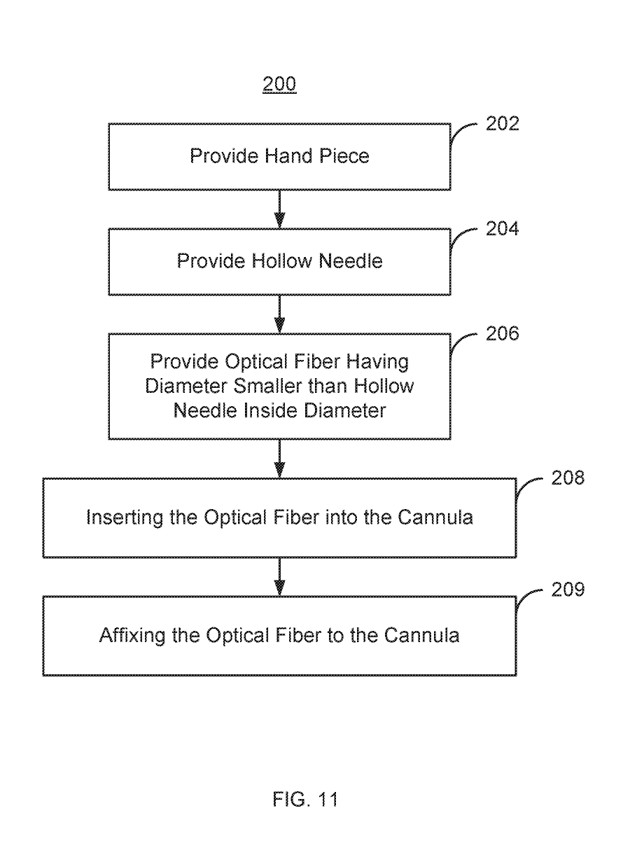

FIG. 11 is a flow chart of an exemplary embodiment of a method 200 for providing an endoilluminator such as the endoilluminator(s) 100, 150, 150A, 150B, 150C and/or 150D. For simplicity, some elements of the method may be omitted, interleaved, and/or combined. The method 200 is also described in the context of the endoilluminator 100. However, the method 200 may be used to form the endoilluminator 150, 150A, 150B, 150C, 150D and/or an analogous endoilluminators.

A hand piece 130 is provided, via 202. The needle 120 is also provided, via 204. Element 204 may include connecting the needle to the handpiece 130. The optical fiber 110 is also provided, via 206. Element 208 may include inserting the optical fiber 110 into the needle 120 (e.g., into channel 122 of needle 120), and element 210 may include affixing the optical fiber to the needle 120 (e.g., through an adhesive). Using the method 200, the endoilluminator 100, 150, 150A, 150B, 150C and/or 150D may be fabricated. Thus, the benefits of one or more of the endoilluminators 100, 150, 150A, 150B, 150C and/or 150D may be achieved.

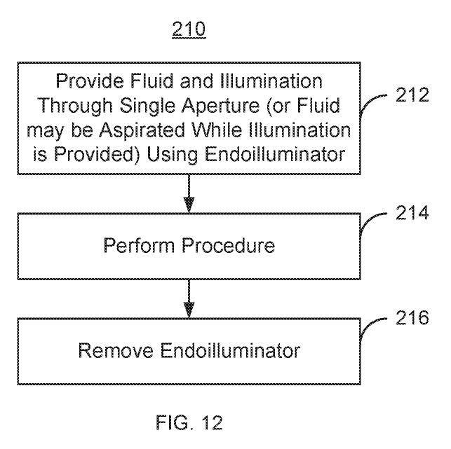

FIG. 12 is a flow chart depicting an exemplary embodiment of a method 210 for assisting a physician during ophthalmic surgery using an endoilluminator such as the endoilluminator 100, 150, 150A, 150B, 150C and/or 150D. For simplicity, some elements of the method may be omitted, interleaved, performed in another order and/or combined. The method 210 is described in the context of ophthalmic surgery and the endoilluminator 100. However, the method 210 may be extended to other types of surgery.

The method may commence after surgery has started. Fluid such as BSS® and illumination are provided (or fluid may be aspirated while illumination is provided) via the endoilluminator 100, via 212. Thus, element 212 may include the surgeon making an incision in the eye of the patient and performing other required tasks. The surgeon may also insert the needle 120 into the incision in patient's eye at 212. Thus, the pressure of the eye may be maintained (or aspirate fluid from the eye) and the operating field illuminated.

The desired procedure is performed, via 214. Thus, portions of the vitreous may be removed. Other procedures may also be performed. For example, membranes may be peeled/dissected and aspirated through the endoilluminator. The needle 120 may then be removed, via 216

Using the method 210 and the endoilluminator 100, 150, 150A, 150B, 150C and/or 150D, ophthalmic surgery may be facilitated and patient safety may be improved. A method and system for providing an endoilluminator, particularly for ophthalmic surgery, have been described. The method and systems have been described in accordance with the exemplary embodiments shown, and one of ordinary skill in the art will readily recognize that there could be variations to the embodiments, and any variations would be within the spirit and scope of the method and system. Accordingly, many modifications may be made by one of ordinary skill in the art without departing from the spirit and scope of the appended claims.

We claim:

1. An endoilluminator, comprising:
a hand piece;
a needle coupled with the hand piece and having a channel therethrough, the channel having an inside diameter, the needle having an outside diameter greater than the inside diameter; and
an optical fiber coupled with the needle, at least a portion of the optical fiber coupled with the needle having an optical fiber diameter of not more than one half the inside diameter;
wherein the optical fiber extends down the needle off of an interior channel wall such that fluid flowing through the needle circumscribes the optical fiber.

2. The endoilluminator of claim 1, wherein the optical fiber diameter is not more than one hundred micrometers.

3. The endoilluminator of claim 2, wherein the optical fiber diameter is not more than fifty micrometers.

4. The endoilluminator of claim 1, further including:
a fluid line coupled with the channel such that fluid may be provided or aspirated through the endoilluminator.

5. The endoilluminator of claim 1, wherein the needle includes an end and the optical fiber includes a tip proximate to the end of the needle.

6. The endoilluminator of claim 5, wherein the tip is selected from a tapered tip, a scattering tip and a flat cleaved tip.

7. The endoilluminator of claim 1, wherein the optical fiber extends down a center of the cannula.

8. The endoilluminator of claim 1, wherein the optical fiber extends off-axis down the cannula.

9. An endoilluminator, comprising:
a hand piece;
a needle coupled with the hand piece, having an end and having a channel therethrough, the channel having an inside diameter, the needle having an outside diameter greater than the inside diameter;
an optical fiber coupled with the needle and having a tip, at least a portion of the optical fiber residing in the channel and having an optical fiber diameter of not more than fifty micrometers, the tip being selected from a tapered tip, a scattering tip and a flat cleaved tip, and
a fluid line coupled with the channel such that fluid may be provided or aspirated through the channel and adjacent to the at least the portion of the optical fiber;
wherein the optical fiber extends down the needle off of a channel wall such that fluid flowing through the needle circumscribes the optical fiber.

10. The endoilluminator of claim 9, wherein the optical fiber diameter is not more than fifty micrometers.

11. The endoilluminator of claim 9, wherein the needle includes an end and the optical fiber includes a tip proximate to the end of the needle, the tip being selected from a tapered tip, a scattering tip and a flat cleaved tip.

12. The endoilluminator of claim 9, wherein the optical fiber extends down a center of the cannula.

13. The endoilluminator of claim 9, wherein the optical fiber extends off-axis down the cannula.

14. A method for treating an ophthalmic condition in an eye of a patient comprising:
infusing or aspirating fluid to or from the eye and providing illumination to an operating field within the eye using an endoilluminator and a single aperture in the eye, the endoilluminator including a hand piece, a needle and an optical fiber, the needle being coupled with the hand piece and having a channel therethrough, the channel having an inside diameter, the needle having an outside diameter greater than the inside diameter, the optical fiber being coupled with the needle, at least a portion of the optical fiber coupled with the needle having an optical fiber diameter of not more than one half the inside diameter, a fluid line being coupled with the channel such that fluid may be provided through the endoilluminator, wherein the optical fiber extends down the needle off of an interior channel wall such that fluid flowing through the needle circumscribes the optical fiber.

15. The method of claim 14, wherein the optical fiber diameter is not more than fifty micrometers.

16. The method of claim 14, wherein the needle includes an end and the optical fiber includes a tip proximate to the end of the needle, the tip being selected from a tapered tip, a scattering tip and a flat cleaved tip.

17. The method of claim 14, wherein the optical fiber extends down a center of the cannula.

18. The method of claim 14, wherein the optical fiber extends off-axis down the cannula.
